(19) United States
(12) Patent Application Publication
(10) Pub. No.: US 2009/0186056 A1
(43) Pub. Date: Jul. 23, 2009

(54) COMPOSITIONS AND METHODS FOR PREVENTING INFECTION

(75) Inventors: George A. Scheele, La Jolla, CA (US); James E. Hildreth, Woodstock, MD (US)

Correspondence Address: KNOBBE MARTENS OLSON & BEAR LLP, 2040 MAIN STREET, FOURTEENTH FLOOR, IRVINE, CA, 92614, US

(73) Assignee: LA JOLLA BIOSCIENCES LLC, La Jolla, CA (US)

(21) Appl. No.: 12/407,733

(22) Filed: Mar. 19, 2009

Related U.S. Application Data
(63) Continuation of application No. 10/637,793, filed on Aug. 8, 2003.
(60) Provisional application No. 60/402,318, filed on Aug. 8, 2002.

(51) Int. Cl. A61K 9/00; A61K 31/724; A61P 31/18

(52) U.S. Cl. 424/402; 514/54; 424/400

(57) ABSTRACT

This invention relates to cholesterol-sequestering agents and methods of using cholesterol-sequestering agents to prevent infection. The compositions of the invention can be used to decontaminate skin and environmental surfaces that are contacted with microorganisms such as envelope viruses.

### CROSS REFERENCE TO RELATED APPLICATIONS

This application claims priority from U.S. Provisional Application No. 60/402,318, filed Aug. 8, 2002. The prior application is incorporated herein by reference in its entirety.

### TECHNICAL FIELD

This invention relates to methods and compositions for preventing infection, and more particularly to methods and compositions using cholesterol-sequestering agents.

### BACKGROUND

Therapeutic approaches have proven largely ineffective for treating infections with envelope viruses such as human immunodeficiency virus (HIV) and other sexually transmitted viral diseases (STDs). In addition, non-sexually transmitted viral diseases such as influenza as well as infections caused by a variety of other microorganisms continue to flourish largely unchecked. The medical community is thus faced with a major need to develop: viricides that destroy human immunodeficiency virus (HIV), herpes simplex virus (HSV) and other causative agents of sexually transmitted diseases (STDs); viricides that destroy non-sexually transmitted viruses causing diseases such as influenza and parainfluenza; and compounds that destroy wide spectrum of infectious microorganisms, such as viruses, bacteria, mycobacteria, fungi, and protozoa.

### SUMMARY

In one aspect, the invention features a composition containing an absorbent material containing a cholesterol-sequestering agent that prevents or reduces viral transmission, wherein the composition is formulated for dermal application to release an amount of the cholesterol-sequestering agent effective to reduce or prevent dermal transmission of an envelope virus to an individual when the composition is contacted to the skin of the individual.

A “cholesterol-sequestering agent” refers to a compound that binds to cholesterol and extracts and depletes cholesterol from a biological membrane such as a plasma membrane or a membrane of an envelope virus. A cholesterol-sequestering agent preferentially extracts cholesterol from lipid rafts present in a biological membrane. The cholesterol-sequestering agent can be, for example, a cyclodextrin. In one example, the cholesterol-sequestering agent is a beta-cyclodextrin such as 2-OH-propyl-beta-cyclodextrin.

The envelope virus can be, for example, a human immunodeficiency virus (HIV) such as HIV-1 or HIV-2; a human herpes virus (HHV) such as HHV1, HHV2, HHV3, HHV4, HHV5, HHV6, HHV7, or HHV8; a hepatitis virus such as hepatitis B virus, hepatitis C virus, or hepatitis D virus; a pox virus such as a small pox virus or molluscum contagiosum virus; an orthomyxovirus such as an influenza virus types A, B, or C; a paramyxovirus such as a mumps virus or a parainfluenza virus type 1, 2, 3, or 4; a human T-cell lymphotropic (HTLV) virus such as HTLV type I or II; a togaviruses such as rubella virus, yellow fever virus, or sinbis virus; ebola virus; or a coronavirus such as severe acute respiratory syndrome (SARS) virus. The envelope virus can be any type or any strain of a given envelope virus. Non-limiting examples of envelope viruses and various types are described herein.

In addition to envelope viruses, the compositions and methods described herein can be used to prevent dermal transmission of a variety of microorganisms, for example, a bacterium (e.g., anthrax or chlamydia), a mycobacterium (e.g., mycobacterium tuberculosis), a virus (e.g., a non-envelope virus, e.g., a protein coated virus such as picorna virus), a fungus, or a protozoan.

The composition can be in any form that allows for the release of the cholesterol-sequestering agent to a surface of the skin. For example, the cholesterol-sequestering agent can be contained in a swab or a towel such as a wipe. In one embodiment, the absorbent material is cotton.

In one embodiment, the envelope virus is a human immunodeficiency virus (HIV). In other embodiments, the envelope virus is a human herpes virus, a hepatitis virus, a pox virus, an influenza virus, a parainfluenza virus, or a human T-cell lymphotropic virus (HTLV).

The invention also features a kit containing a plurality of compositions, e.g., swabs or wipes, described herein. For example, a kit can contain at least 10, 50, 100, 500, 1,000, 10,000, or more of the compositions described herein. In some embodiments, the compositions contained in the kits are sterilized.

In another aspect, the invention features a glove containing an interior surface and an exterior surface, wherein the interior surface or the exterior surface of the glove is coated with an amount of a cholesterol-sequestering agent described herein effective to reduce or prevent dermal transmission of an envelope virus to an individual wearing the glove.

The cholesterol-sequestering agent contained in the glove can be, for example, a cyclodextrin. In one example, the cholesterol-sequestering agent is a beta-cyclodextrin such as 2-OH-propyl-beta-cyclodextrin. The envelope virus can be any envelope virus described herein. In one embodiment, the interior surface of the glove is coated with the cholesterol-sequestering agent. In another embodiment, the exterior surface of the glove is coated with the cholesterol-sequestering agent. In yet another embodiment, the interior surface and the exterior surface of the glove are coated with the cholesterol-sequestering agent. The interior and/or exterior of the glove can optionally be coated with a powder containing the cholesterol-sequestering agent.

In one embodiment, the envelope virus is a human immunodeficiency virus (HIV). In other embodiments, the envelope virus is a human herpes virus, a hepatitis virus, a pox virus, an influenza virus, a parainfluenza virus, or a human T-cell lymphotropic virus (HTLV). The invention also features a kit containing a plurality of gloves containing a coating of a cholesterol-sequestering agent, as described herein. For example, a kit can contain at least 10, 50, 100, 500, 1,000, 10,000, or more of the gloves described herein. In some embodiments, the gloves contained in the kits are sterilized.

In another aspect, the invention features a method of reducing or preventing viral transmission, the method including the steps of: selecting an individual engaged in an activity associated with an elevated risk of exposure to an envelope virus, wherein the activity is not a sexual or drug use-related activity; and contacting a skin surface of the individual with a composition containing an amount of a cholesterol-sequestering agent effective to reduce or prevent dermal transmission of the envelope virus to the individual.

In one embodiment, the individual is a medical worker, e.g., a person that handles blood, blood products, or some other biological sample containing human material. In another embodiment, the individual is a soldier. For example, the method can be used to prevent the infection of a soldier with a pox virus.

In one embodiment, an extremity, e.g., the hands, of the individual is contacted with the composition. For example, the composition can be a glove containing an interior surface and an exterior surface, wherein the interior surface and/or the exterior surface of the glove is coated with an amount of the cholesterol-sequestering agent effective to reduce or prevent dermal transmission of the envelope virus to the individual. In another example, the composition contains an absorbent material containing the cholesterol-sequestering agent, wherein the composition is contacted to the skin surface of the individual and releases an amount of the cholesterol-sequestering agent effective to reduce or prevent transmission of the envelope virus to the individual.

In one embodiment, the composition is contacted to a portion of the skin containing a lesion or an irritation. For example, the composition can be contacted to a portion of the skin containing a lesion resulting from a puncture of the skin with a medical instrument. In one embodiment, the medical instrument is a needle or a scalpel.

In another embodiment, the composition contains a cream containing the cholesterol-sequestering agent. In another embodiment, the composition contains a spray containing the cholesterol-sequestering agent. In another embodiment, the composition contains a powder containing the cholesterol-sequestering agent.

In another aspect, the invention features a method of treating an environmental surface, the method including the steps of: identifying a surface that has been exposed to blood or a blood product; and contacting the surface with a composition containing an amount of a cholesterol-sequestering agent effective to reduce the amount of an envelope virus, if present, on the surface. An environmental surface refers to a surface that is not made of living cells, e.g., a surface that does not contain the skin of an individual. An environmental surface includes, for example, tables, floors, chairs, equipment, instruments, and the like.

In one embodiment, the surface is located in a medical facility. For example, the surface can be located in a patient care facility or in a medical laboratory.

A composition used according to the present method can be any composition described herein. In one embodiment, the composition contains an absorbent material containing the cholesterol-sequestering agent, wherein the composition is contacted to the surface and releases an amount of the cholesterol-sequestering agent effective to reduce the amount of an envelope virus, if present, on the surface.

In another embodiment, the composition contains a spray containing the cholesterol-sequestering agent. In another embodiment, the composition contains a powder containing the cholesterol-sequestering agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### DETAILED DESCRIPTION

The present invention provides methods and compositions for preventing an infection by microorganisms such as envelope viruses. The methods and compositions of the invention make use of a cholesterol-sequestering agent that can have one or more of several possible effects on a microorganism. For some microorganisms such as envelope viruses, a cholesterol-sequestering agent may cause the lysis of the virus. By removing cholesterol from a viral membrane, a cholesterol-sequestering agent not only disrupts the ordered structure of membrane elements, but further destroys the integrity of the membrane itself leading to disruption of the viral membrane and leakage of viral contents, an irreversible process that fully inactivates the viral particle. In some instances, a cholesterol-sequestering agent may block the uptake of an intracellular pathogen by blocking endocytosis in a cell. Accordingly, a cholesterol-sequestering agent can be used to prevent dermal transmission of microorganisms and/or to decontaminate a surface that has come into contact with blood or a blood product that contains an envelope virus.

### Cholesterol-Sequestering Agent

Any cholesterol-sequestering agent can be used in the methods and compositions of the invention. As described herein, a cholesterol-sequestering agent binds to cholesterol and extracts and depletes cholesterol from a biological membrane, such as a plasma membrane or a membrane of an envelope virus. A cholesterol-sequestering agent preferentially extracts cholesterol from lipid rafts present in a biological membrane.

Examples of cholesterol-sequestering agents include compounds such as cyclodextrins, nystatin, and filipin. Cyclodextrins include both naturally occurring cyclodextrins, e.g., alpha, beta, and gamma cyclodextrins, as well as derivatives of naturally occurring cyclodextrins. Non-limiting examples of derivatives of naturally occurring cyclodextrins include derivatives of beta cyclodextrin such as hydroxypropyl beta cyclodextrin, carboxy-methyl beta cyclodextrin, and methyl beta cyclodextrin. For a detailed description on cyclodextrins and derivatives thereof, see, e.g., Parrish, M. A. “Cyclodextrins—a Review.” Sterling Organics Ltd., Newcastle-Upon-Tyne, England; and cyclodex.com.

Beta cyclodextrin, a simple sugar ring structure containing seven alpha (1-4) glucopyranose units, has the ability to rapidly extract cholesterol from lipid rafts, thereby disrupting their ordered membrane structure. As a result of cholesterol removal, lipid rafts are dispersed in the plane of the membrane and the mechanisms responsible for entry and exit of envelop viruses from target cells are abolished.

Beta cyclodextrin has a particularly high affinity for cholesterol. When used at concentrations ranging from 5-100 mM, 2-HP-BCD removes membrane cholesterol within minutes. At the molecular level, beta cyclodextrin resembles a toroid or cup-like structure with openings at both the top and bottom. The toroid structure contains hydrophilic groups on the exterior surface and hydrophobic groups on the interior surface. The hydrophylic groups confer solubility in aqueous medium while the hydrophobic groups form the hydrophobic pocket that binds the cholesterol.

Hydroxypropyl beta cyclodextrin is an example of a derivative of beta cyclodextrin that can be used in the methods of the invention. Hydroxypropyl beta cyclodextrin is a partially substituted poly(hydroxpropyl)ether of beta cyclodextrin. The basic closed circular structure of beta cyclodextrin is maintained in hydroxypropyl beta cyclodextrin. The glycosidic oxygen forming the bond between the adjacent glucose monomers and the hydrogen atoms lining the cavity of the cyclodextrin impart an electron density and hydrophobic character to the cavity. Organic compounds interact with the walls of the cavity to form inclusion complexes. The hydroxyl groups and the hydroxypropyl groups are on the exterior of the molecule and interact with water to provide the increased aqueous solubility of the hydroxypropyl beta cyclodextrin and the complexes made with the hydroxypropyl beta cyclodextrin. For a detailed description of the structure of hydroxypropyl beta cyclodextrin, see, e.g., Muller et al. (1986) “Hydroxypropyl-B-cyclodextrin derivatives: Influence of average degree of substitution on complexing ability and surface activity” J. Pharm. Sci. 75.

### Compositions Containing Cholesterol-Sequestering Agents

As detailed herein, by removing cholesterol from the membrane of an envelope virus, a cholesterol-sequestering agent not only disrupts the ordered structure of the membrane elements, but further destroys the integrity of the membrane itself leading to disruption of viral membranes and leakage of viral contents into the medium, an irreversible process that can fully inactivate the viral particle.

Envelope viruses that can be targeted using the methods and compositions described herein include, but are not limited to: a human immunodeficiency virus (HIV) such as HIV-1 or HIV-2; a human herpes virus (HHV) such as HHV1, HHV2, HHV3, HHV4, HHV5, HHV6, HHV7, or HHV8; a hepatitis virus such as hepatitis B virus, hepatitis C virus, or hepatitis D virus; a pox virus such as a small pox virus or molluscum contagiosum virus; an orthomyxovirus such as an influenza virus types A, B, or C; a paramyxovirus such as a mumps virus or a parainfluenza virus type 1, 2, 3, or 4; a human T-cell lymphotropic virus (HTLV) such as HTLV type I or II; a togaviruses such as rubella virus, yellow fever virus, or sinbis virus; ebola virus; or a coronavirus such as severe acute respiratory syndrome (SARS) virus.

In addition to envelope viruses, the methods and compositions described herein can be used to prevent dermal transmission of a variety of microorganisms, for example, a bacterium (e.g., anthrax or chlamydia), a mycobacterium (e.g., mycobacterium tuberculosis), a virus (e.g., a non-envelope virus, e.g., a protein coated virus such as picorna virus), a fungus, or a protozoan.

The methods and compositions described herein can be used to prevent clinical conditions that result from an infection by an envelope virus, including but not limited to AIDS (HIV infection), certain cancers (caused by HTLV types I and II), fever blisters or cold sores (Herpes labialis; HHV1 infection), genital herpes (Herpes genitalis; HHV2 infection), chicken pox (HHV3 infection), herpes zoster or shingles (HHV3 infection), mononucleosis (HHV4 infection), cytomegalovirus infection (HHV infection), Kaposi's Sarcoma (HHV8 infection), German measles (rubella virus infection), or severe acute respiratory syndrome (SARS virus infection).

A variety of compositions can be formulated to contain an amount of a cholesterol-sequestering agent effective to reduce or prevent dermal transmission of an envelope virus to an individual. Accordingly, the skin of an individual can be protected from viral transmission by applying a composition described herein to the skin. The cholesterol-sequestering agent can be contained in, for example, creams, lotions, ointments, gels, liquids, sprays, powders, or absorbent materials. The composition can be applied to the skin before and/or after contacting the skin with a material (e.g., blood or a blood product) that contains or potentially contains an infective envelope virus.

Hands, other body extremities, and/or any exposed part of an individual can be contacted with the composition following the individual's exposure to blood or a blood product. According to such a method, the cholesterol-sequestering agent renders an envelope virus present on the individual's skin uninfective, thereby reducing the likelihood that the individual will become infected by the envelope virus. The methods encompass the application of the composition to both apparently healthy skin of the individual (e.g., skin lacking any observable lesions) as well as the application of the composition to an area of skin containing a lesion or an area of skin that is irritated and/or is a site of inflammation. For a skin lesion resulting from a medical procedure carried out in the presence of blood or a blood products (e.g., a lesion resulting from a needle puncture or scalpel nick), the administration of the composition directly to the lesion can decrease the likelihood that the individual will become infected by the virus.

The composition can be contacted with the skin in the form of, for example, a cream, lotion, gel, spray, or powder. In addition, the composition can be contained in an absorbent material, wherein the composition is formulated to release an amount of the cholesterol-sequestering agent effective to reduce or prevent dermal transmission of the envelope virus to the individual when the composition is contacted to the skin of the individual. Examples of compositions containing an absorbent material include, but are not limited to, swabs or towels such as wipes. Such absorbent materials can be used to effectively clean an area of skin. Compositions used in such methods can contain additional antiviral and/or anti-microbial compounds in addition to the cholesterol-sequestering agent.

In addition to contacting skin with the composition following exposure to blood or a blood product, hands and/or other portions of the body of an individual can also or in the alternative be contacted with the composition before exposure to blood or a blood product. Such preemptive action can be useful to provide an individual with protection in case an unexpected exposure to an envelope virus occurs, e.g., during the course of a medical procedure. The cholesterol-sequestering agent can be applied to the skin using any formulation, including but not limited to the various formulations described herein.

In addition to the compositions described herein, an individual can also be protected against infection by an envelope virus by the use of a physical barrier that prevents the virus from contacting the skin. Examples of physical barriers frequently used in the medical community include gloves, gowns, masks, and eyewear. Any of these physical barriers can be formulated to also contain a cholesterol-sequestering agent described herein, so as to reduce or prevent the dermal transmission of an envelope virus that breaches such a physical barrier and reaches the skin of an individual. For example, gloves can be used that contain a coating (an interior and/or exterior coating) of a composition containing a cholesterol-sequestering agent. Suitable gloves include any type that is used in medical and/or research environments. For example, the gloves can be made of latex and can optionally be of the disposable variety. The coating of the composition can be in the form of a powder, cream, lotion, gel, or other suitable form.

A composition containing a cholesterol-sequestering agent can also include a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

In addition to methods of contacting skin with a cholesterol-sequestering agent to reduce or prevent dermal transmission of an envelope virus, the compositions described herein can also be used to treat an environmental surface, e.g., an inanimate surface in a medical or research facility. In general an environmental surface that has been exposed to blood or a blood product (or has been potentially exposed to blood or a blood product) is contacted with a composition containing an amount of a cholesterol-sequestering agent effective to reduce the amount of an envelope virus, if present, on the environmental surface. The cleaning of a surface with a composition described herein can be incorporated as part of a routine procedure carried out between separate uses of a given environmental surface. For example, an environmental surface can be cleaned with a composition described herein during the interval between two patients or two sets of patient material being brought into contact with the same surface. The carrying out of the method decontaminates the environmental surface so as to reduce or eliminate the possibility that a patient or biological material that later contacts the surface will also come into contact with an envelope virus.

### Kits

A variety of compositions are described in the present application that contain an amount of a cholesterol-sequestering agent effective to reduce or prevent dermal transmission of an envelope virus to an individual. These compositions are particularly useful for the inactivation of envelope viruses that contact the skin of an individual or that reside on a surface that has been contaminated by blood or a blood product.

The compositions can be prepared, e.g., by mass production techniques, to generate large numbers of substantially identical compositions, each of which may be subject to single or multiple use by an individual. For example, gloves containing an amount of a cholesterol-sequestering agent effective to reduce or prevent dermal transmission of an envelope virus to an individual wearing the glove can be prepared and packaged in containers, e.g., boxes, containing at least 10, 50, 100, 500, 1,000, 10,000, or more of such coated gloves. In another example, an absorbent composition (e.g., a swab or a wipe) formulated to release an amount of a cholesterol-sequestering agent effective to reduce or prevent dermal transmission of an envelope virus to an individual can also be prepared and packaged in containers, e.g., boxes, containing at least 10, 50, 100, 500, 1,000, 10,000, or more of such absorbent compositions. Instructions can be included with such containers (e.g., in the form of a paper included in the container or a writing on the container) that instruct the user that the composition reduces or prevents infection by an envelope virus described herein.

Kits as described herein can also contain a dispenser that facilitates the removal of individual compositions from a container.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A glove comprising an interior surface and an exterior surface, wherein the interior surface is coated with an amount of a cholesterol-sequestering agent effective to reduce or prevent dermal transmission of an envelope virus to an individual wearing the glove.

2. The glove of claim 1, wherein the exterior surface of the glove is coated with the cholesterol-sequestering agent.

3. The glove of claim 1, wherein the glove is coated with a powder comprising the cholesterol-sequestering agent.

4. The glove of claim 1, wherein the cholesterol-sequestering agent is a cyclodextrin.

5. The glove of claim 4, wherein the cyclodextrin is a beta-cyclodextrin.

6. The glove of claim 5, wherein the beta-cyclodextrin is 2-OH-propyl-beta-cyclodextrin.

7. The glove of claim 1, wherein the envelope virus is a human immunodeficiency virus (HIV).

8. The glove of claim 1, wherein the envelope virus is a human herpes virus, a hepatitis virus, a pox virus, an influenza virus, a parainfluenza virus, or a human T-cell lymphotropic virus (HTLV).

9. A kit comprising a plurality of gloves according to claim 1.

10. The kit of claim 11, wherein the kit comprises at least 10 of the gloves.

11. The kit of claim 11, wherein the kit comprises at least 50 of the gloves.

12. The kit of claim 11, wherein the kit comprises at least 100 of the gloves.

13. A composition comprising an absorbent material comprising a cholesterol-sequestering agent that prevents or reduces viral transmission, wherein the composition is formulated for dermal application to release an amount of the cholesterol-sequestering agent effective to reduce or prevent dermal transmission of an envelope virus to an individual when the composition is contacted to the skin of the individual.

14. The composition of claim 13, wherein the composition is a swab.

15. The composition of claim 13, wherein the composition is a wipe.

16. The composition of claim 13, wherein the absorbent material is cotton.

17. The composition of claim 13, wherein the cholesterol-sequestering agent is a cyclodextrin.

18. The composition of claim 17, wherein the cyclodextrin is a beta-cyclodextrin.

19. The composition of claim 18, wherein the beta-cyclodextrin is 2-OH-propyl-beta-cyclodextrin.

20. The composition of claim 13, wherein the envelope virus is a human immunodeficiency virus (HIV).

21. The composition of claim 13, wherein the envelope virus is a human herpes virus, a hepatitis virus, a pox virus, an influenza virus, a parainfluenza virus, or a human T-cell lymphotropic virus (HTLV).

22. A kit comprising a plurality of compositions according to claim 13.

23. The kit of claim 22, wherein the kit comprises at least 10 of the compositions.

24. The kit of claim 22, wherein the kit comprises at least 50 of the compositions.

25. The kit of claim 22, wherein the kit comprises at least 100 of the compositions.

26. A method of reducing or preventing viral transmission, the method comprising:
selecting an individual engaged in an activity associated with an elevated risk of exposure to an envelope virus, wherein the activity is not a sexual or drug use-related activity; and
contacting a skin surface of the individual with a composition comprising an amount of a cholesterol-sequestering agent effective to reduce or prevent dermal transmission of the envelope virus to the individual.

27. The method of claim 26, wherein the individual is a medical worker.

28. The method of claim 27, wherein the medical worker is a blood handler.

29. The method of claim 26, wherein the individual is a soldier.

30. The method of claim 26, wherein a hand of the individual is contacted with the composition.

31. The method of claim 30, wherein the composition is a glove comprising an interior surface and an exterior surface, wherein the interior surface of the glove is coated with the amount of the cholesterol-sequestering agent effective to reduce or prevent dermal transmission of the envelope virus to the individual.

32. The method of claim 26, wherein the composition comprises an absorbent material comprising the cholesterol-sequestering agent, and wherein the composition is contacted to the skin surface of the individual and releases an amount of the cholesterol-sequestering agent effective to reduce or prevent transmission of the envelope virus to the individual.

33. The method of claim 32, wherein the composition is contacted to a portion of the skin comprising a lesion or an irritation.

34. The method of claim 33, wherein the composition is contacted to a portion of the skin comprising a lesion resulting from a puncture of the skin with a medical instrument.

35. The method of claim 34, wherein the medical instrument is a needle or a scalpel.

36. The method of claim 26, wherein the composition comprises a cream comprising the cholesterol-sequestering agent.

37. The method of claim 26, wherein the composition comprises a spray comprising the cholesterol-sequestering agent.

38. The method of claim 26, wherein the composition comprises a powder comprising the cholesterol-sequestering agent.

39. A method of treating an environmental surface, the method comprising:
identifying a surface that has been exposed to blood or a blood product; and
contacting the surface with a composition comprising an amount of a cholesterol-sequestering agent effective to reduce the amount of an envelope virus, if present, on the surface.

40. The method of claim 39, wherein the surface is located in a medical facility.

41. The method of claim 40, wherein the medical facility is a patient care facility.

42. The method of claim 40, wherein the medical facility is a medical laboratory.

43. The method of claim 39, wherein the composition comprises an absorbent material comprising the cholesterol-sequestering agent, and wherein the composition is contacted to the surface and releases an amount of the cholesterol-sequestering agent effective to reduce the amount of an envelope virus, if present, on the surface.

44. The method of claim 39, wherein the composition comprises a spray comprising the cholesterol-sequestering agent.

45. The method of claim 39, wherein the composition comprises a powder comprising the cholesterol-sequestering agent.
